(19) United States
(12) Patent Application Publication
(10) Pub. No.: US 2007/0219146 A1
(43) Pub. Date: Sep. 20, 2007

(54) Synergistic Pharmaceutical And/Or Neutraceutical Flavanoid Composition For Management Of Diabetes Mellitus

(76) Inventors: Sunil Bhaskaran, Pune (IN); Vishwaraman Mohan, Pune (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 11/669,197

(22) Filed: Jan. 31, 2007

(30) Foreign Application Priority Data
Feb. 6, 2006 (IN) 01949/MUM/2006

(51) Int. Cl. A61K 31/7048; A61K 31/522; A61K 31/353

(52) U.S. Cl. 514/27; 514/263.31; 514/456

(57) ABSTRACT

The present invention relates to a synergistic pharmaceutical and/or neutraceutical flavanoid composition for management of Diabetes Mellitus, said composition comprising polyphenol of concentration ranging between 85 to 95% (w/w) GAE, theobromine of concentration ranging between 1 to 5% (w/w), and moisture content ranging between 0.5 to 10% (v/w).

### FIELD OF THE INVENTION

The present invention relates to a purified flavanoid composition derived from plant matter specifically from Theobroma Cocoa intended for kinder and gentler management of blood glucose in subjects affected by Diabetes Mellitus. This composition can be used as a pharmaceutical, nutraceutical or as functional food ingredient for this purpose.

### BACKGROUND OF THE INVENTION

Diabetes Mellitus is the most common endocrine disease. This disease is characterized by poor regulation of blood glucose levels in human beings. Blood glucose is the source of energy for basic cell functions. This glucose is driven to the cell by insulin, which is secreted by pancreas. Diabetes Mellitus is caused by inadequate insulin secretion by the pancreas or the resistance generated by cells to the insulin. Therefore, this disease is characterized by a metabolic abnormality. Diabetes is a major metabolic disorder in which the body does not produce or properly use insulin that is characterized by hyperglycemia, glycosuria, hyperlipidemia, negative nitrogen balance and sometimes ketonemia. Diabetes is one of the most common diseases affecting human population today.

In India as per WHO reports, about 60 million people would be suffering from diabetes by the year 2025. This would put India as the no. 1 country in the world affected by this epidemic disorder. Diabetes among urban Indians had increased from 11.8% in 1995 to 13.2% in 2000. This disorder strikes people during the most productive stage of their life. With the recent studies conducted and published, it appears that this is caused more by lifestyle, food habits and genetic predisposition for the disease. This is particularly true in urban areas wherein the lifestyle results in people exercising inadequately or tending to eat more processed ready to eat food. This in turn has led to obesity and later to onset of diabetes. Diabetes Mellitus is not curable. Presently, this disorder can be managed by taking popular drugs available in the market place. These drugs fall into the following categories:—
i. Pancreatic stimulators:—This class of drugs helps to stimulate the pancreas, leading to increased secretion of insulin. This addresses the diabetes caused by inadequate insulin secretion.
ii. Insulin sensitisors:—This category of drugs improves the cell's sensitivity to the presence of insulin, thereby improving uptake of glucose into the cells, leading to better blood sugar control.
iii. Insulin:—This is exogenously supplemented in the case of people suffering from both type I and type II diabetes.

As mentioned earlier, diabetes is a lifestyle disease and cannot be cured. The current therapies available therefore only offer a blood sugar management mechanism. As diabetes is a chronic, long duration disease, these drugs need to be taken on a sustained basis. Currently, available synthetic drugs suffer from concomitant side effects caused due to long duration of usage. Literature survey indicates that cardio vascular mortality was higher in patients with oral hypoglycemics than in those treated with diet and exercise alone or with insulin. Sulphonylureas cause hypoglycemia as a side effect. Biguamides cause lactic acidosis. Oral hypoglycemic drugs cause GIT irritation, weight gain, hypertension, etc. On continuous and constant exertion, the diabetic person is liable for pancreatic fatigue. In addition, it is also seen that many of the existing drugs available lead to drug resistance in patients with long durations of use.

As mentioned earlier, the long-term complications of diabetes are more damaging. This is caused by the spikes in blood sugar in patients during the day. Increased blood sugar even for short periods leads to glycosylation of Haemoglobin. And Glycosylated Haemoglobin causes long-term irreversible damages to eyes, kidneys, nerves and blood vessels.

Complications of diabetes: A wide spread pathological change is thickening of capillary basement membrane, increase in vessel wall matrix and cellular proliferation resulting in vascular complications like lumen narrowing, early atherosclerosis, sclerosis of glomerular capillaries, retinopathy, neuropathy and peripheral vascular insufficiency. The level of glycosylated hemoglobin (HbA1c) is also increased in diabetes and is taken as an index of protein glycoslyation. It reflects the state of glycaemia over the preceding 2-3 months. As such there is no drug available for the treatment of diabetic complications.

Consequently, the need of the hour is to develop safe and efficacious drugs that can help in management of blood sugar in diabetes mellitus patients. This drug should lend itself for long term use without any side affects and without developing resistance. Therefore, there is an urgent requirement for a kinder and gentler medication for chronic management of diabetic condition. The proposed invention will offer this solution for management of blood sugar in subjects affected by Diabetes Mellitus for long-term duration.

### DESCRIPTION OF RELATED ART

Use of Cocoa as food, drink and medicine originated in Meso America within the Mayan and Olmec civilization. Cocoa, derived from the seeds of Theobroma Cacao, can be a rich source of flavanoids, especially the flavan-3-ols and their related oligomers. The presence of flavanoids in notable concentrations in Cocoa has triggered numerous studies on their potential health benefits. Flavanoids have been reported to have significant antioxidant and potential anti-carcinogenic activities.

Cocoa (Theobroma Cocoa) is well known for its application in confectionery. Although Cocoa butter is the most commonly used ingredient of the fruit of Theobroma Cocoa, the fruit, particularly its Kernel, contains other ingredients, which are very interesting.

One such ingredient is Theobromine. This ingredient is becoming popular for its acceleration of metabolism and weight loss property. Today, it is getting increasingly incorporated into novel nutraceutical products meant for weight management. However, this is a cardiac stimulant and can be harmful if taken regularly. Besides, it is reported that Theobromine has reproductive toxicity as well.

Another significant ingredient of the Cocoa kernel is flavanoids. These flavanoids are not lipid soluble and hence do not come along with Cocoa butter. They are left behind in the kernel powder, when the butter is extracted. These flavanoids are very powerful anti-oxidants and have interesting properties in free radical scavenging, leading to increased cardiovascular health.

Scientists at the United States Department of Agriculture (USDA) have studied these flavanoids after characterising them. They are found to play significant roles in certain Cytokine secretion regulation, leading to control in histamine secretion. Recent human feeding studies have confirmed that the monomeric flavanols in Cocoa can be absorbed after the ingestion of chocolate. In addition to being anti-oxidants, the flavanols and their related oligomers isolated from Cocoa may have marked anti-inflammatory properties, mediated in part, through inhibition of peroxynitrate formation.

Flavanoid compounds are present in all aerial parts of plants with high concentrations found in the skin, bark and seeds. Such compounds are also found in beverage of botanical substances such as tea, wine, coffee and cocoa. The flavanoids are member of a larger family of compounds called polyphenols. These compounds contain more than one phenolic —OH group on the respective benzene ring.

Procyanidins are particular class of colorless flavanoid compounds with a general structure as follows:

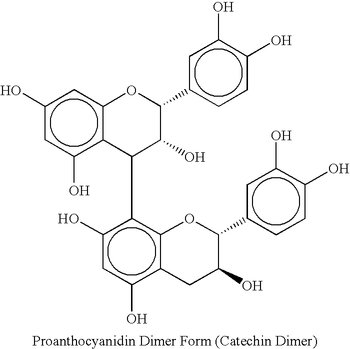

Proanthocyanidins yield anthocyanin upon hydrolysis with inorganic acids, and anthocyanins are responsible for the reddish color of grapes, flowers and other plant materials.

Bate—Smith and Bate—Smith Lerner (1954) systematically investigated a broad variety of flavanoid compounds. The acid hydrolysis of proanthocyanidins, in addition to anthocyanidins, yields catechin. Catechin is the building unit of dimmers, trimmers and oligomers.

Jean Claude Stoclet et al. has reviewed the beneficial effects of various flavanoids in “Vascular Protection by dietary flavanoids” in European Journal of Pharmacology 500 (2004) 299-313. They have confirmed that the consumption of flavanoid rich foods such as fruits, vegetables and beverages derived from plants such as cocoa, wine, and tea may offer a beneficial diet in terms of cardiovascular protection. They have explained the mechanism of action of some of these flavanoids in offering improvement in endothelial function and inhibition of angiogenesis.

We are discussing some of the medicinal effects of the Cocoa in the following. Effect of Cocoa flavanols and their related oligomers on the secretion of interleukin-5 in Peripheral Blood Mononuclear cells. Journal of Medicinal Food. Volume 5, Number 1, 2002. T. K. Mao, VAN DE WATER et al. University of California, Davis.

In the present study, it is examined whether selected FLO (flavanols and their related oligomers) fractions isolated from Cocoa (monomer through decamer) modulate IL-5 protein secretion from resting and phytohemagglutinin (PHA)—stimulated peripheral blood mononuclear cells (PBMC). Although FLO fractions were unstimulatory for IL-5 secretion in resting cells, PHA—induced IL-5 release from PBMC was markedly affected by certain FLO fractions. The monomeric and small oligomeric (dimer and trimer) fractions enhanced PHA stimulation by 50%, 54% and 43% respectively. In contrast, the larger oligomeric fractions (hexamer through decamer) inhibited IL-5 release in the range of 18% to 39%; the tetramer and pentamer showed intermediate effects. The increment in IL-5 suggests that FLO may preferentially stimulate immunoglobulin A. It is suggested that in the oral cavity, this could result in reduction in the risk for dental caries and periodontal disease. This work offers additional data for consideration of the health benefits of dietary FLO from a variety of foods, including those benefits associated specifically with consumption of some Cocoas and chocolates.

Chocolate Procyanidins decrease the leukotriene—prostacyclin ratio in humans and human aortic endothelial cells. Am. J. Clin. Nutr 2001; 73-36-40. Derek D. Schramm, Janice F. Wang et al. University of California, Davis.

Flavanoidic phytochemicals inhibit vascular and inflammatory processes that contribute to disease. These effects are hypothesized to result from flavanoid—mediated alterations in cellular eicosanoid synthesis. The objective was to determine and compare the ability of Cocoa procyanidins to alter eicosanoid synthesis in human subjects and cultured human aortic endothelial cells. Data from this short-term investigation support the concept that certain food—derived flavanoids can favorably alter eicosanoid synthesis in humans, providing a plausible hypothesis for a mechanism by which they can decrease platelet activation in humans.

Cocoa, flavanols and Cardiovascular Risk. Br. J. Cardiol 2004; 11:379-86—Norman Hollenberg et al. Brigham and Women's Hospital Boston.

There has been a long-standing interest in the relation between what we eat and cardiovascular risk. Over the years, attention has been given to calories, total fat, saturated fat, cholesterol, omega-3 polyunsaturated fatty acids, trans fatty acids, folic acid, antioxidants and, most recently, flavanols. Flavanol concentrations can be moderately high in a number of foods that have been associated with a reduction in cardiovascular risk, including red wine as well as black and green tea. Some Cocoa and chocolate products are extraordinarily rich in flavanols but, as with other flavanol-containing foods, certain post-harvesting and processing procedures can have a striking influence on the flavanol content of chocolate and Cocoa.

Endothelial dysfunction, with a consequent reduction in nitric oxide production, has achieved a central conceptual role in the pathogenesis of atherosciorosis and coronary artery disease, diabetes mellitus and hypertension. Recent evidence that flavanol-rich cocoa activates vascular nitric oxide synthesis in endothelia, raises an interesting possibility of therapeutic potential.

Effects of Cocoa Extracts on Endothelium—Dependent Relaxation.—J. Nutr. 130:2105 S-2108S, 2000. The American Society for Nutritional Sciences: 2000. Malina Karim, Killie Mc Cormick et al. University of California, Davis.

The aim of this study was to examine the effects of procyanidins derived from Cocoa on vascular smooth muscle. Two hypotheses were tested: 1) extracts of Cocoa, which are rich in procyanidins, cause endothelium-dependent relaxation (EDR), and 2) extracts of Cocoa activate endothelial nitric oxide synthase (NOS). The experiments were carried out on aortic rings obtained from New Zealand White rabbits. The polymeric procyanidins (tetramer through decamer of catechin) caused an EDR. In addition, the Ca²⁺-dependent NOS activity, measured by the L-arginine to L-Citrulline conversion assay, was significantly increased in aortic endothelial cells exposed to polymeric procyanidins, whereas monomeric compounds had no such effect. These findings demonstrate that polymeric procyanidins cause an EDR that is mediated by activation of NOS.

Ruzaidi, Amin et al. The effect of Malaysian cocoa extract on glucose levels and lipid profiles in Diabetic Rats (Journal of Ethno pharmacology 98 (2005); 55-60. They have administered a crude alcoholic extract of defatted cocoa beans to diabetic rats at the rate of 1%, 2% and 3% of daily feed. They found that 3% administration resulted in significant reduction in blood sugar level. It is interesting to note that 2% level of feeding did not have any significant reduction in blood sugar level. This study does not lead to a composition, which can be used for chronic administration in diabetic subjects, because of the following inherent difficulties.
i) This is a crude extract containing about 28% of Polyphenol and about 8-10% Theobromine and other constituents of Cocoa. The dosage proposed at 1%, 2%, and 3% of the feed is at 220 mg/kg, 440 mg/kg and 660 mg/kg of the crude extract as per the experiment reported. This is fairly large dose and significant effect is seen only at 3% dose viz., 660 mg/kg.
ii) The crude alcohol extract contains the alkaloid Theobromine at least at 8-10% level. This means that there is a Theobromine dosing of 66 mg/kg body weight for 660 mg/kg administration of the crude drug. Theobromine is cardiac stimulant and cannot be given regularly at this dose due to adverse side effects. Besides, Theobromine is having reproductive toxicity as per published studies

There have been various studies which confirm that higher level of flavanoid dosing leads to inhibition of iron absorption leading to reduction in RBC count and Hemoglobin. This is a very serious adverse reaction.

James D. Cook et al. has investigated the effect of red wine on non hemi-iron absorption in human in American Journal of Nutrition 1995: 61; 800-4. They confirm that higher concentration of flavanoids lead to significant inhibition of iron absorption. Rossander and Hallberg have studied Iron absorption and phenolic compounds in European Journal of Clinical Nutrition 1989, 43, 547-558.

Therefore this study does not lead to a useful composition for chronic administration for blood sugar reduction as the dose is very high leading to serious iron absorption inhibition combined with ill effects of the presence of Theo bromine in large quantity.

As this is a crude Alcoholic extract with 28% Polyphenol, the balance 72% are other materials derived from Cocoa. It is not clear as to which compound is having action on blood sugar reduction. Therefore it is not possible to devise a scientific method to commercially produce this material for consistent bioactivity in the absence of knowledge about the marker compound for hypoglycemia.

Therefore, there is a need to obtain pure marker compound fractions of cocoa and purify them to high levels of purity for testing this fraction for its efficacy and safety for management of blood sugar so that this marker compound fraction can be identified, repeatability of action can be established and this can be made commercially in a consistent way so as to get bioactivity.

### OBJECTS OF THE PRESENT INVENTION

The main object of the present invention is to develop a synergistic pharmaceutical and/or neutraceutical flavanoid composition for management of Diabetes Mellitus. Another main object of the present invention is to develop a method for sustained management of Diabetes Mellitus in a subject in need thereof.

Yet another object of the present invention is to develop a method for management of type II diabetes.

Still another object of the present invention is to develop a process for preparing a synergistic pharmaceutical and/or neutraceutical flavanoid composition for management of Diabetes Mellitus.

### SUMMARY OF THE PRESENT INVENTION

A synergistic pharmaceutical and/or neutraceutical flavanoid composition for management of Diabetes Mellitus, said composition comprising polyphenols of concentration ranging between 85 to 95% (w/w), theobromine of concentration ranging between 1 to 5% (w/w), optionally along with pharmaceutically acceptable additives, a method for sustained management of Diabetes Mellitus in a subject in need thereof, said method comprising administering pharmaceutically effective amount of a synergistic pharmaceutical and/or neutraceutical flavanoid composition comprising polyphenols of concentration ranging between 85 to 95% (w/w); theobromine of concentration ranging between 1 to 5% (w/w), optionally along with pharmaceutically acceptable additives to the subject, and a process for preparing a synergistic pharmaceutical and/or neutraceutical flavanoid composition for management of Diabetes Mellitus, said composition comprising polyphenol of concentration ranging between 85 to 95% (w/w) and theobromine of concentration ranging between 1 to 5% (w/w), optionally along with pharmaceutically acceptable additives, said process comprising steps of:
a. extracting cocoa beans using alkyl ester to obtain extract,
b. drying the extract to obtain solvent free mass,
c. extracting the dried mass with an aqueous alcoholic solvent to obtain extract,
d. filtering and concentrating the extract to obtain pasty mass,
e. dissolving the pasty mass in water and filtered to obtain clear solution,
f. passing the clear solution through an adsorbent column, followed by washing the column with water,
g. eluting the column with an pure alcohol to obtain elluent, and
h. reducing the alcohol content of elluent to obtain the composition as pure flavanoid powder.

### DETAILED DESCRIPTION OF THE PRESENT INVENTION

A synergistic pharmaceutical and/or neutraceutical flavanoid composition for management of Diabetes Mellitus, said composition comprising polyphenols of concentration ranging between 85 to 95% (w/w), theobromine of concentration ranging between 1 to 5% (w/w), optionally along with pharmaceutically acceptable additives.

In an embodiment of the present invention, the concentration of polyphenol is about 90% w/w.

In another embodiment of the present invention, the concentration of theobromine is about 4% w/w.

In yet another embodiment of the present invention, wherein the additives are selected from a group comprising granulating agents, binding agents, lubricating agents, disintegrating agents, coloring agents, flavoring agents, coating agents, plasticizers, preservatives, suspending agents, emulsifying agents and spheronization agents.

In still another embodiment of the present invention, wherein the composition has moisture content ranging between 0.5 to 10% (v/w), preferably about 3% v/w. In yet another embodiment of the present invention, wherein the composition comprises two major polyphenols at Rf 0.87 and 0.77; a minor polyphenol at Rf 0.71; and theobromine at Rf 0.14.

One more embodiment of the present invention, a method for sustained management of Diabetes Mellitus in a subject in need thereof, said method comprising administering pharmaceutically effective amount of a synergistic pharmaceutical and/or neutraceutical flavanoid composition comprising polyphenols of concentration ranging between 85 to 95% (w/w); theobromine of concentration ranging between 1 to 5% (w/w), optionally along with pharmaceutically acceptable additives to the subject.

In still another embodiment of the present invention, wherein the concentration of polyphenol is preferably about 90% w/w.

In yet another embodiment of the present invention, wherein the concentration of theobromine is preferably about 4% w/w.

In still another embodiment of the present invention, wherein the composition has moisture content ranging between 0.5 to 10% (v/w), preferably about 3% v/w.

In yet another embodiment of the present invention, wherein the additives are selected from a group comprising granulating agents, binding agents, lubricating agents, disintegrating agents, coloring agents, flavoring agents, coating agents, plasticizers, preservatives, suspending agents, emulsifying agents and spheronization agents.

In still another embodiment of the present invention, wherein the subject is an animal including humans.

In yet another embodiment of the present invention, wherein the subject is administered said composition at daily dosage of 1 mg/kg to 25 mg/kg body weight.

In still another embodiment of the present invention, wherein the composition is safe for administration.

In yet another embodiment of the present invention, wherein the diabetes is type II diabetes.

In still another embodiment of the present invention, wherein the composition is administered orally.

Further embodiment of the present invention, a process for preparing a synergistic pharmaceutical and/or neutraceutical flavanoid composition for management of Diabetes Mellitus, said composition comprising polyphenol of concentration ranging between 85 to 95% (w/w) and theobromine of concentration ranging between 1 to 5% (w/w), optionally along with pharmaceutically acceptable additives, said process comprising steps of:
a) extracting cocoa beans using alkyl ester to obtain extract,
b) drying the extract to obtain solvent free mass,
c) extracting the dried mass with an aqueous alcoholic solvent to obtain extract,
d) filtering and concentrating the extract to obtain pasty mass,
e) dissolving the pasty mass in water and filtered to obtain clear solution,
f) passing the clear solution through an adsorbent column, followed by washing the column with water,
g) eluting the column with an pure alcohol to obtain elluent, and
h) reducing the alcohol content of elluent to obtain the composition as pure flavanoid powder.

In yet another embodiment of the present invention, wherein cocoa beans are obtained from Theobroma cocoa.

In still another embodiment of the present invention, wherein alkyl ester is selected from a group comprising methyl acetate, ethyl acetate, propyl acetate and butyl acetate.

In yet another embodiment of the present invention, wherein the extract is dried at room temperature.

In still another embodiment of the present invention, wherein the alcohol of aqueous alcoholic solvent is selected from a group comprising methyl alcohol, ethyl alcohol, isopropyl alcohol, and butanol.

In yet another embodiment of the present invention, wherein the aqueous alcoholic solvent is a mixture of ethyl alcohol and water in the ratio ranging between 4:3 to 19:1.

In still another embodiment of the present invention, wherein extracting the dried mass at temperature ranging between 15 to 45° C. for time duration ranging between 6 to 15 hours.

In yet another embodiment of the present invention, wherein the water is de-mineralized water.

In still another embodiment of the present invention, wherein the extract is filtered with filter paper.

In yet another embodiment of the present invention, wherein the adsorbent column is a non-ionic polymeric adsorbent column.

In still another embodiment of the present invention, wherein pure alcohol is selected from a group selected from methyl alcohol, ethyl alcohol, isopropyl alcohol, and butanol.

In yet another embodiment of the present invention, wherein reducing the alcohol content of elluent with vacuum evaporation.

In still another embodiment of the present invention, wherein the additives are selected from a group comprising granulating agents, binding agents, lubricating agents, disintegrating agents, coloring agents, flavoring agents, coating agents, plasticizers, preservatives, suspending agents, emulsifying agents and spheronization agents.

In yet another embodiment of the present invention, wherein the composition has moisture content ranging between 0.5 to 10% (v/w), preferably about 3% v/w.

The isolation and purification of the pure flavanoids from the cocoa beans involve the following steps:
(I) Extraction of the cocoa beans using an ester solvent which may be an alkyl ester to remove the fats and other alkaloids present in the coco beans, which are not relevant to the present study.
(II) Ethyl esters comprising of alkyl groups of carbon ranging from one carbon atom to four-carbon atom. Examples are methyl acetate, ethyl acetate, propyl acetate and Butyl acetate. The acetates are selected because of their selectivity against flavanoid extraction.
(III) Generally, anthocynidins and colours are not soluble in ester solvents.
(IV) After defatting the mass with alkyl ester solvents, it is dried at room temperature in a forced draft air circulation drier to achieve solvent free mass.
(V) The dried mass is packed in a suitable solid liquid extractor of counter current flow of solvent against gravity or a packed column co-current flow system.
(VI) The solvent used for extraction is an aqueous alcoholic solvent preferably a mixture of ethyl alcohol and water in the ratio of 70:30 to a maximum ratio of 95:5 over a period ranging from 8 hrs to 12 hrs, preferably 8 hrs, at temperature ranging from 20° C. to 40° C., preferably at 35° C.
(VII) The alcohol chosen can be from any of the following; Methyl Alcohol, Ethyl Alcohol, Isopropyl Alcohol and Butanol
(VIII) The clear extract is filtered to remove any suspended particles and is concentrated at a temperature ranging between 45° C. to 60° C., preferably at 50° C. under vacuum to get a pasty mass.
(IX) The above mass is dissolved in De-mineralized water (10 volumes of the weight of pasty mass) and filtered to remove any solids.
(X) The clear solution is passed through an adsorbent column consisting of Amberlite XAD-16, a non-ionic polymeric adsorbent having 800 m²/gm surface area. The adsorbed material is desorbed using pure alcohol ranging from methyl alcohol, ethyl alcohol, Isopropyl alcohol and Butanol.
(XI) This elute is subjected to vacuum evaporation to reduce the alcohol content by vacuum evaporation.
(XII) The concentrated slurry can be dried by using a vacuum dryer or a spray dryer to get a pure flavanoid powder. This flavanoid powder contains no residual solvent as the drying conditions are properly controlled to eliminate all the residual solvents.
(XIII) This flavanoid powder is tested for the polyphenol content to standardize this by Folin C method.
(XIV) This flavanoid composition is tested on Thin Layer chromatography for identifying marker compounds and is found to contain three flavanoids and one Theobromine. Out of this two flavanoids account for more than 90% of the total flavanoid.
(XV) This flavanoid is subjected to HPLC testing to check and confirm the Theobromine content is less than 5%.
(XVI) The composition thus obtained is having approximately 93% polyphenol content, 4% Theobromine content, 3% Moisture.
(XVII) This standardized flavanoid composition is administered to diabetic mice to check acute level activity. It is found that 50 mg/kg admnistration gave the best effect which is comparable to the effects produced by 10 mg/kg Glyburide.
(XVIII) This standardized composition of flavanoid is administered to Diabetic mice to check the Sub acute effect for 28 days. A surprising invention is made here, that lower closes are more effective. The best effective dose is 100 mg per kg body weight. 50 mg per kg also had efficacy. Higher doses at 250-mg/kg body weight is not effective at all.
(XIX) This standardized composition is administered to 6 treatment naïve and newly diagnosed diabetic subjects for 3 months. They have reported a significant reduction in blood sugar. A dosage of 300 mg/kg is well tolerated during this 3-month time.

The invention is further elaborated with the help of following examples. However, these examples should not be construed to limit the scope of the instant invention.

Experiment No 1:

1000 grams of pulverized cocoa beans with an average size ranging from 16 mesh passing, is soaked in ethyl acetate and poured in to an extractor having a perforated bottom sieve of the 200 mesh sieve. The bottom elluent is recycled again and again over the packed mass to achieve effective extraction for a period of 8 hrs. The elluent is discarded and the mass is removed out of the extractor and dried in a forced draft Oven at 30° C. After removal of solvent by drying, the mass was again packed in the extractor. The packed mass is extracted with 5 liters of a solvent mixture comprising of 70:30 mixture of ethyl alcohol and D.M water and the extract is recycled over the bed for about 8 hrs at 35° C. to achieve efficient extraction. The extract is filtered though filter paper and concentrated at 40° C. under vacuum to get 50 grams of pasty material. The isolated material is redissolved in 3 liters of D.M water and passed through a column consisting of 100 ml of the adsorbent resin Amberlite XAD-16 after the completion of the feed. The column was thoroughly washed with D.M. water free of adhering substances and the elluent is neutral. The column is further eluted with pure Ethyl alcohol. The elluent is concentrated and diluted with water and spray dried to get a free flowing powder. The final weight is 10 grams and the Folin C value of this is 92.3%.

Experiment No 2

1000 gms of pulverized coco beans with an average size ranging from 16 mesh Passing. Is soaked in ethyl acetate and poured in to an extractor having a perforated Bottom sieve of the 200 mesh sieve. The bottom elluent is recycled again and again over the packed mass to achieve effective extraction for a period of 8 hrs. The elluent is discarded and the mass is removed out of the extractor and dried in a forced draft Oven at 30° C. After removal of solvent by evaporation, the mass was again packed in the extractor. The packed mass is extracted with 5 liters of a solvent mixture comprising of 60:40 mixture of ethyl alcohol and D.M water. The extract is recycled over the bed for about 8 hrs at 35° C. to achieve efficient extraction. The extract is filtered though filter paper and concentrated at 40° C. under vacuum to get 70 Gms of pasty material. The isolated material is redissolved in 3 liters of D.M water and passed through a column consisting of 100 ml of the adsorbent resin Amberlite XAD-16 after the Completion of the feed. The column was thoroughly washed with D.M. water free of adhering substances and the elluent is neutral. The column is further eluted with pure Ethyl alcohol & the collected elluent is concentrated and diluted with water and spray dried to get a free flowing powder. The final weight is 9.5 Gms and the Folin C value is 94.7%.

Experiment No 3:

1000 gms of pulverized coco beans with an average size ranging from 16 mesh Passing. is soaked in ethyl acetate and poured in to an extractor having a perforated Bottom sieve of the 200 mesh sieve. The bottom elluent is recycled again and again over the packed mass to achieve effective extraction for a period of 8 hrs. The elluent is discarded and the mass is removed out of the extractor and dried in a forced draft Oven at 30° C. After removal of solvent by evaporation, the mass was again packed in the extractor. The packed mass is extracted with 5 liters of a solvent mixture comprising of 80:20 mixture of Isopropyl alcohol and D.M water. The extract is recycled over the bed for about 8 hrs at 35° C. to achieve efficient extraction. The extract is filtered though filter paper and concentrated at 40° C. under vacuum to get 46 Gms of pasty material. The isolated material is redissolved in 3 liters of D.M water and passed through a column consisting of 100 ml of the adsorbent resin Amberlite XAD-16 after the completion of the feed. The column was thoroughly washed with D.M. water free of adhering substances and the elluent is neutral. The column is further eluted with pure Ethyl alcohol & the collected elluent is concentrated and diluted with water and Spray dried to get a free flowing powder. The yield is 8 gms and the Folin C value is 91.9%.

Experiment No 4:

1000 gms of pulverized coco beans with an average size ranging from 16 mesh Passing. is soaked in ethyl acetate and poured in to an extractor having a perforated Bottom sieve of the 200 mesh sieve. The bottom elluent is recycled again and again over the packed mass to achieve effective extraction for a period of 8 hrs. The elluent is discarded and the mass is removed out of the extractor and dried in a forced draft Oven at 30° C. After removal of solvent by evaporation, the mass was again packed in the extractor. The packed mass is extracted with 5 liters of a solvent mixture comprising of 90:10 mixture of Isopropyl alcohol. And D.M water the extract is recycled over the bed for about 8 hrs at 35° C. to achieve efficient extraction. The extract is filtered though filter paper and concentrated at 40° C. under vacuum to get 60 Gms of pasty material. The isolated material is redissolved in 3 liters of D.M water and passed through a column consisting of 100 ml of the adsorbent resin Amberlite XAD-16 after the completion of the feed. The column was thoroughly washed with D. M. Water free of adhering substances and the elluent is neutral. The column is further eluted with pure isopropyl alcohol & the collected elluent is concentrated and diluted with water and spray dried to get a free flowing powder. The final dry weight is 8 gms and the Folin C value is 91.6%.

Experiment No 5:

Swiss Albino mice (male) in the weight range 25 to 30 grams were studied for acute affect. They were made diabetic by the admnistration of Alloxan (with admnistration of 70 mg/kg Alloxan and in 48 hours the animals develop diabetes). As the mice became diabetic and the blood sugar was above 250 mg/kg, they were grouped into four groups of six each. Each group was administered by the test drug at 25 mg/kg, 50 mg/kg 100 mg/kg and 250 mg/kg body weight. One group was administered a positive control drug glyburide at 10 mg/kg of body weight. Blood samples were drawn at 2 hour, 4 hour, 6 hour and 24 hour intervals for all mice. The blood sugar pattern is studied. It has been found out that the best results are obtained for a dose of 50 mg/kg body weight at 2 hours after administration and it increases up to the 6^{th} hour. Good result is obtained for 100 mg/kg drug. However, there is no significant effect for higher dose at 250 mg/kg and lower dose at 25 mg/kg. The effect of 50 mg/kg compared very well with the effect of positive control drug glyburide at 10 mg/kg body weight.

This is an interesting finding that the hypoglycemic effect is best for a dose ranging between 50 mg to 100 mg/kg of body weight. Besides, it is also found that higher dose have little or no effect.

Acute Study of Cocoa Flavanoid
Summary of readings (Blood sugar level)

| | | Normal | 2 | 4 | 6 | 24 |
|---|---|---|---|---|---|---|
| 10 | mg/kg(Glyburide) | 374.33 | 317.50 | 266.16 | 149.50 | 228.66 |
| 25 | mg/kg | 426.83 | 391.91 | 388.00 | 362.14 | 397.03 |
| 50 | Mg/kg | 253.72 | 146.88 | 85.06 | 90.05 | 208.79 |
| 100 | Mg/kg | 333.17 | 273.17 | 139.33 | 251.33 | 251.33 |
| 250 | Mg/kg | 332.83 | 332.17 | 260.50 | 259.17 | 324.33 |

Acute Study of Cocoa Flavanoid
Summary in Percentage (Blood sugar level)

| | | Normal | 2 | 4 | 6 | 24 |
|---|---|---|---|---|---|---|
| 25 | mg/kg | 426.83 | 8% | 9% | 15% | 7% |
| 50 | Mg/kg | 253.72 | 42% | 66% | 65% | 18% |
| 100 | Mg/kg | 333.17 | 18% | 58% | 25% | 25% |
| 250 | Mg/kg | 332.83 | 0% | 22% | 22% | 3% |
| 10 | mg/kg(Glyburide) | 374.33 | 15% | 29% | 60% | 39% |

Experiment No 6:

Swiss Albino mice (Male) in the weight range of 25 to 30 gram were studied for sub acute effect for 28 days. The mice were made diabetic by admnistration of Alloxan. As the mice became diabetic and the blood sugar was above 250 mg/kg they were chosen for the study. 4 groups were made. One group was given 50 mg/kg, 2^{nd} group 100 mg/kg, 3^{rd} group 250 mg/kg and the 4^{th} group positive control glyburide at 10 mg/kg. Treatment was given for 28 days.

A very interesting observation was made that the best effect was produced by the dose of 100 mg/kg body weight. The effect started by 7^{th} day and increased up to 28^{th} day. 50 mg/kg showed significant effect. Unlike acute study, the sub acute study has confirmed that 100 mg/kg given the most optimal blood sugar reduction. This effect was sustained after the removal of treatment on 35^{th} day.

Sub Acute Study

Sub Acute Study of Cocoa Flavanoid
Summary of readings (Blood sugar level)

| | Normal | 7 | 14 | 21 | 28 |
|---|---|---|---|---|---|
| 50 mg/kg | 253.72 | 178.28 | 186.41 | 173.82 | 140.29 |
| 100 mg/kg | 323.02 | 192.72 | 141.43 | 154.31 | 113.38 |
| 250 mg/kg | 340.15 | 362.79 | 316.75 | 341.81 | 270.36 |
| Glyburide | 396.09 | 323.46 | 265.46 | 170.82 | 137.90 |

Sub Acute Study of Cocoa Flavanoid
Summary in percentage (Blood sugar level)

| | 7 | 14 | 21 | 28 |
|---|---|---|---|---|
| 50 mg/kg | 30% | 27% | 31% | 45% |
| 100 mg/kg | 40% | 56% | 52% | 65% |
| 250 mg/kg | −7% | 7% | 0% | 21% |
| Glyburide | 18% | 33% | 57% | 65% |

Experiment No 7:

The analysis of Cocoa Flavanoids from Theobroma Cocoa is based on the following reference.

Singleton, V. L. and Rossi, J. L. 1965 Colorimetry of total phenolics with phosphomolybdic—phosphotungstic acid reagents. Am J. Enol. Vitic. 16:144-158

100 mg of the flavanoids derived from Theobroma Cocoa was taken in water and made up to 100 ml in a standard flask. Transfer 5 ml of this solution into 100 ml flask and make up to volume using water. Pipette out 1 ml of the above solution into nessler tube. Add 15 ml water followed by 1 ml Folin—Ciocalteu Reagent. Mix well. Add 3 ml of 20% Sodium carbonate solution and place in 40° C. constant temperature bath for 20 minutes. Immediately after the reaction, cool the test tubes in an ice bath to room temperature after zeroing the spectrophotometer with the water blank. Measure the absorbance at 755 nm using 1 cm cell. Compare this with a standard solution of Gallic Acid prepared in the same way and calculate the percentage of Flavanoids as % W/W Gallic Acid Equivalent (GAE). Give moisture correction. The material analyzed in this way gave 85% to 92% total Flavanoids.

[image]

All the spots are visible at 254 nm

Detection limit fot UV is 10 ppm & Detection limit for folin-c spray is 50 ppm. This TLC fingerprinting confirms the presence of two major polyphenols at Rf at 0.87 and 0.77 respectively and a minor polyphenol at Rf 0.71 followed by Theobromine at Rf 0.14.

HPLC of the polyphenol confirmed 3 polyphenol peaks and one Theobromine peak. Two polyphenols accounting for 90% content on the basis of area purity of Chromatogram

Experiment No 8:

The test drug at a dose of 300 mg/kg was orally given in a hard gelatin capsule form to 6 treatment naïve newly diagnosed diabetic subjects in the weight range of 60 to 72 kg of body weight. These subjects have elevated fasting and postprandial blood sugar after diet and exercise. These are candidates who may have to start with oral hypoglycemic agents soon. The study was done for a period of 3 months with visits at each month. Prior to the study, fasting blood sugar, postprandial blood sugar, kidney function test, liver function test and blood hematology were tested. At the end of the study all the tests were repeated. During visit 1 and visit 2, only blood tests for fasting and postprandial sugar were taken.

It is found that these subjects tolerated the drug very well without any side effects. The parameter of kidney function, liver function and blood hematology remained almost the same without any significant changes.

It has been found that all the subjects reported significant reduction in blood sugar. There is an average reduction of 39 mg/dl in fasting blood sugar and 27.6 mg/dl reduction in postprandial blood sugar during the study. The drug is effective at a dose of 300 mg in reducing blood sugar in diabetic subjects.

Dosage of 300 mg per day was orally given to 6-treatment naïve newly diagnosed diabetic patients (3 men and 3 women).

| | | | Blood Sugar Level | | | |
|---|---|---|---|---|---|---|
| | | | | | After adminis- | |
| | | | Before adminis- | | tration of Cocoa | |
| | | | tration of Cocoa | | Flavanoid for 3 | |
| | | | Flavanoid | | month at 300 mg/day | |
| Patient | | | | Post | | Post |
| No | Gender | Weight | Fasting | Prandial | Fasting | Prandial |
| 1 | Male | 72 | 141 | 211 | 100 | 151 |
| 2 | Male | 71 | 135 | 213 | 90 | 153 |
| 3 | Male | 71 | 142 | 205 | 98 | 139 |
| 4 | Female | 60 | 133 | 202 | 95 | 149 |
| 5 | Female | 65 | 131 | 191 | 99 | 138 |
| 6 | Female | 64 | 135 | 195 | 101 | 148 |

| | | | | |
|---|---|---|---|---|
| AVERAGE | 136.17 | 173.9 | 97.17 | 146.3 |

The composition of instant invention is synergistic in nature. The composition shows extraordinary activity in the management of Diabetes Mellitus. The activity of the composition is well beyond the expectations of the inventors and is also significantly more than the additive effect of the individual components of the composition. Thus, the activity is surprising in nature and is thus both novel and inventive in nature. Lastly, the Industrial Application of the composition is well-established as the problem of Diabetes Mellitus is widespread and is affecting children also in a big way. Therefore, the instant composition is a leap forward towards the management of this menace.

What is claimed:

1. A synergistic pharmaceutical and/or neutraceutical flavanoid composition for management of Diabetes Mellitus, said composition comprising polyphenols of concentration ranging between 85 to 95% (w/w), theobromine of concentration ranging between 1 to 5% (w/w), optionally along with pharmaceutically acceptable additives.

2. The synergistic composition as claimed in claim 1, wherein the concentration of polyphenol is about 90% w/w.

3. The synergistic composition as claimed in claim 1, wherein the concentration of theobromine is about 4% w/w.

4. The synergistic composition as claimed in claim 1, wherein the additives are selected from a group comprising granulating agents, binding agents, lubricating agents, disintegrating agents, coloring agents, flavoring agents, coating agents, plasticizers, preservatives, suspending agents, emulsifying agents and spheronization agents.

5. The synergistic composition as claimed in claim 1, wherein the composition has moisture content ranging between 0.5 to 10% (v/w), preferably about 3% v/w.

6. The synergistic composition as claimed in claim 1, wherein the composition comprises two major polyphenols at Rf 0.87 and 0.77; a minor polyphenol at Rf 0.71; and theobromine at Rf 0.14.

7. A method for sustained management of Diabetes Mellitus in a subject in need thereof, said method comprising administering pharmaceutically effective amount of a synergistic pharmaceutical and/or neutraceutical flavanoid composition comprising polyphenols of concentration ranging between 85 to 95% (w/w); theobromine of concentration ranging between 1 to 5% (w/w), optionally along with pharmaceutically acceptable additives to the subject.

8. The method for management of Diabetes Mellitus as claimed in claim 7, wherein the concentration of polyphenol is preferably about 90% w/w.

9. The method for management of Diabetes Mellitus as claimed in claim 7, wherein the concentration of theobromine is preferably about 4% w/w.

10. The method for management of Diabetes Mellitus as claimed in claim 7, wherein the composition has moisture content ranging between 0.5 to 10% (v/w), preferably about 3% v/w.

11. The method for management of Diabetes mellitus as claimed in claim 7, wherein the additives are selected from a group comprising granulating agents, binding agents, lubricating agents, disintegrating agents, coloring agents, flavoring agents, coating agents, plasticizers, preservatives, suspending agents, emulsifying agents and spheronization agents.

12. The method for management of Diabetes Mellitus as claimed in claim 7, wherein the subject is an animal including humans.

13. The method for management of Diabetes Mellitus as claimed in claim 7, wherein the subject is administered said composition at daily dosage of 1 mg/kg to 25 mg/kg body weight.

14. The method for management of Diabetes Mellitus as claimed in claim 7, wherein the composition is safe for administration.

15. The method for management of Diabetes Mellitus as claimed in claim 7, wherein the diabetes is type II diabetes.

16. The method for management of Diabetes Mellitus as claimed in claim 7, wherein the composition is administered orally.

17. A process for preparing a synergistic pharmaceutical and/or neutraceutical flavanoid composition for management of Diabetes Mellitus, said composition comprising polyphenol of concentration ranging between 85 to 95% (w/w) and theobromine of concentration ranging between 1 to 5% (w/w), optionally along with pharmaceutically acceptable additives, said process comprising steps of:
a. extracting cocoa beans using alkyl ester to obtain extract,
b. drying the extract to obtain solvent free mass,
c. extracting the dried mass with an aqueous alcoholic solvent to obtain
extract,
d. filtering and concentrating the extract to obtain pasty mass,
e. dissolving the pasty mass in water and filtered to obtain clear solution, passing the clear solution through an adsorbent column, followed by washing the column with water,
f. eluting the column with an pure alcohol to obtain elluent, and
g. reducing the alcohol content of elluent to obtain the composition as pure flavanoid powder.

18. The process as claimed in claim 17, wherein cocoa beans are obtained from Theobroma cocoa.

19. The process as claimed in claim 17, wherein alkyl ester is selected from a group comprising methyl acetate, ethyl acetate, propyl acetate and butyl acetate.

20. The process as claimed in claim 17, wherein the extract is dried at room temperature.

21. The process as claimed in claim 17, wherein the alcohol of aqueous alcoholic solvent is selected from a group comprising methyl alcohol, ethyl alcohol, isopropyl alcohol, and butanol.

22. The process as claimed in claim 17, wherein the aqueous alcoholic solvent is a mixture of ethyl alcohol and water in the ratio ranging between 4:3 to 19:1.

23. The process as claimed in claim 17, wherein extracting the dried mass at temperature ranging between 15 to 45⁰ C. for time duration ranging between 6 to 15 hours.

24. The process as claimed in claim 17, wherein the water is de-mineralized water.

25. The process as claimed in claim 17, wherein the extract is filtered with filter paper.

26. The process as claimed in claim 17, wherein the adsorbent column is a non-ionic polymeric adsorbent column.

27. The process as claimed in claim 17, wherein pure alcohol is selected from a group selected from methyl alcohol, ethyl alcohol, isopropyl alcohol, and butanol.

28. The process as claimed in claim 17, wherein reducing the alcohol content of elluent with vacuum evaporation.

29. The process as claimed in claim 17, wherein the additives are selected from a group comprising granulating agents, binding agents, lubricating agents, disintegrating agents, coloring agents, flavoring agents, coating agents, plasticizers, preservatives, suspending agents, emulsifying agents and spheronization agents.

30. The process as claimed in claim 17, wherein the composition has moisture content ranging between 0.5 to 10% (v/w), preferably about 3% v/w.
